(19) United States
(12) Patent Application Publication
(10) Pub. No.: US 2026/0219130 A1
(43) Pub. Date: Jul. 30, 2026

(54) SYSTEMS, APPARATUS, AND METHODS FOR LENS METROLOGY

(71) Applicant: Johnson & Johnson Surgical Vision, Inc., Irvine, CA (US)

(72) Inventors: Usiel Malave-Robles; Régis Gengler, Groningen (NL); Magnus Akerstrom, Jacksonville, FL (US); Mihai State, Groningen (NL); John Scott, Inverness (GB)

(21) Appl. No.: 19/039,733

(22) Filed: Jan. 28, 2025

(51) Int. Cl. G01M 11/02; G01N 21/958; B65G 54/02

(52) U.S. Cl. CPC G01M 11/0214; G01M 11/0235; G01M 11/0292; G01N 21/958; B65G 54/02; G01N 2021/9583

(57) ABSTRACT

An automated apparatus for measuring characteristics of an intraocular lens is disclosed. The automated apparatus may include a first robotic assembly configured to place an intraocular lens into a lens holding assembly and a second robotic assembly configured to place the lens holding assembly in a lens carrier. The automated apparatus may also include a first automated station for filling the lens carrier with a solution and a second automated station for performing measurements on the intraocular lens carried by the lens carrier. Further, the automated apparatus may include a third automated station for removing the solution from the lens carrier. In addition, the automated apparatus may include a third robotic assembly configured to remove the lens holding assembly from the lens carrier and a fourth automated station configured to dry the intraocular lens.

### FIELD OF INVENTION

The present disclosure is directed to lens metrology, and more particularly, automated systems for measuring characteristics of intraocular lenses.

### BACKGROUND

This background description is provided for the purpose of generally presenting the context of the disclosure. Unless otherwise indicated herein, material described in this section is neither expressly nor impliedly admitted to be prior art to the present disclosure or the appended claims.

Ophthalmic lenses, such as intraocular lenses, may be made by first forming a button from a sheet of a specialized polymer. Alternatively, the button may be formed by cast molding, where a monomer material is deposited in a mold having a cavity defined between opposing mold parts. To prepare a button using such mold parts, an uncured polymer may be placed between a front mold part and a back mold part. The front and back mold parts are brought together to shape the lens according to desired lens parameters. The lens formulation may be subsequently cured, for example by exposure to heat and light, thereby forming a button. Following the curing process, the mold parts are separated and the button is removed from the mold parts. The button may then be formed into an intraocular lens by machining or lathing.

Ophthalmic lenses, such as intraocular lenses or contact lenses, may also be made by cast molding techniques, in which a monomer material is deposited in a mold having a cavity defined between opposing mold parts. To prepare a lens using such mold parts, an uncured polymer may be placed between a front mold part and a back mold part. The front mold part and the back mold part are typically formed via injection molding techniques wherein melted plastic is forced into highly machined steel tooling with at least one surface of optical quality. The front and back mold parts are brought together to shape the lens according to desired lens parameters. The lens formulation may be subsequently cured, for example by exposure to heat and light, thereby forming a lens. Following the curing process, the mold parts are separated and the lens is removed from the mold parts.

Cast molding of ophthalmic lenses, such as intraocular lenses, has been successful for high volume runs of a limited number of lens sizes and powers. However, the nature of the injection molding processes and equipment may make it difficult to form custom lenses specific to a particular patient's eye or a particular application.

Systems for forming customized lenses via the use of voxel based lithographic techniques have also been developed. An important aspect of these techniques is that a lens is produced in a manner where one of two lens surfaces is formed in a free form fashion without cast molding, lathing or other tooling. A free formed surface and base may include a free-flowing fluent media included in the free formed surface. This combination results in a device sometimes referred to as a lens precursor. Fixing radiation and hydration treatments may typically be utilized to convert a lens precursor into an ophthalmic lens or an intraocular lens.

A freeform lens created by any of these methods may need to be measured in order to ascertain the physical parameters of the lens. Therefore, methods and systems are needed for automatically measuring an intraocular lens formed from a precursor and other processes.

### SUMMARY

The present disclosure is directed to embodiments relating to lens metrology. The embodiments provide automated systems configured to inspect and measure characteristics and/or parameters of intraocular lenses. For example, the automated systems may inspect intraocular lenses for image quality, diopter power metrics, and cosmetic defects. The automated systems may also control the automated transfer of intraocular lenses among a plurality of automated stations for handling and processing the intraocular lenses to avoid the necessity for direct manual handling of the intraocular lenses. For example, the automated systems may include a conveyance system for transporting and manipulating the intraocular lenses including placing the intraocular lenses on multiple stations to process manufacturing orders (including identification of a lens model, cylinder power, etc.) In some embodiments, the conveyance system may include a non-contact system, such as a magnetic levitation transport system, to reduce or eliminate debris due to friction or contact with other surfaces. As such, the automated systems may enable the intraocular lenses to be measured without physically damaging or contaminating the intraocular lenses, losing the intraocular lenses, or allowing the intraocular lenses to invert or roll over in a lens carrier.

Further, the automated systems may include embedded control loops enabling reactive corrections of environmental factors (e.g., temperature, water dosage, particle contamination etc.) and may enable product flow adjustments to account for out of calibration components and/or automated processing or handling stations. The automated systems may also include control loops for product and data management (e.g., pass/fail analysis, handling re-measurements, handling measurements of multiple orders simultaneously, etc.). Additionally, the automated systems may quickly and efficiently identify the intraocular lenses that meet certain quality metrics and/or verification standards.

In one aspect, an automated apparatus for measuring characteristics of an intraocular lens is disclosed. The automated apparatus may include a first robotic assembly configured to select the intraocular lens from a tray and to place the intraocular lens into a lens holding assembly. The automated apparatus may also include a second robotic assembly configured to assemble a lens carrier from a plurality of components. The second robotic assembly may be configured to place the lens holding assembly in the lens carrier. The automated apparatus may include a transport system for transporting the lens carrier to and from a plurality of stations. The lens carrier may be placed on the transport system by the second robotic assembly. Further, the automated apparatus may include a first automated station for receiving the lens carrier and filling the lens carrier with a solution and a second automated station for receiving the lens carrier and performing measurements on the intraocular lens carried by the lens carrier. In addition, the automated apparatus may include a third automated station for removing the solution from the lens carrier. The automated apparatus may also include a third robotic assembly configured to remove the lens holding assembly from the lens carrier and a fourth automated station configured to receive the lens holding assembly from the third robotic assembly and to dry the intraocular lens and the individual lens carrier components.

In some embodiments, the second robotic assembly of the automated apparatus may be configured to flip or invert the lens holding assembly prior to placing the lens holding assembly in the lens carrier. Further, the fourth automated system of the automated apparatus may be configured to flip or invert the lens holding assembly to its original orientation or an inverted position. Additionally, the lens carrier may be filled with a temperature-controlled solution.

In another aspect, a method for performing measurements on an intraocular lens is disclosed. The method may include selecting the intraocular lens from a carrier, placing the intraocular lens into a lens holding assembly, and assembling a lens carrier from a plurality of components. The method may also include placing the lens holding assembly in the lens carrier and filling the lens carrier with a solution. Further, the method may include performing measurements on the intraocular lens carried by the lens carrier, removing the solution from the lens carrier, and removing the lens holding assembly from the lens carrier. In addition, the method may include drying the intraocular lens and placing the intraocular lens in a first container or a second container based on the measurements of the intraocular lens.

In some embodiments, the method may include flipping or inverting the lens holding assembly prior to placing the lens holding assembly in the lens carrier. Further, method may include flipping or inverting the lens holding assembly and/or lens to its original orientation or an inverted position after drying the lens. Additionally, the lens carrier may be filled with a temperature-controlled solution.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

FIG. 1 illustrates a perspective view of a system for lens metrology, according to an exemplary embodiment;

FIG. 2 illustrates a front elevation view of the system of FIG. 1;

FIG. 3 illustrates a top view of the system of FIG. 1;

FIG. 4 illustrates a block diagram of a dosing station according to an exemplary embodiment;

FIG. 5 illustrates schematic diagram of a metrology station, according to an exemplary embodiment;

FIG. 6 illustrates a block diagram of a purging station, according to an exemplary embodiment; and

FIG. 7 illustrates a block diagram of a method, according to an exemplary embodiment.

### DETAILED DESCRIPTION

Before explaining the embodiments in detail, it should be noted that the present disclosure is not limited in its application or use to the details of construction and arrangement of parts illustrated in the accompanying drawings and description, because the illustrative embodiments may be implemented or incorporated in other embodiments, variations and modifications, and may be practiced or carried out in various ways. Furthermore, unless otherwise indicated, the terms and expressions employed herein have been chosen for the purpose of describing the embodiments of the present disclosure for the convenience of the reader and are not for the purpose of limitation.

The present disclosure is directed to systems relating to lens metrology. The embodiments provide automated systems configured to inspect and measure characteristics and/or parameters of intraocular lenses. For example, the automated systems may inspect intraocular lenses for image quality, diopter power metrics, and cosmetic defects. The automated systems may also control the automated transfer of intraocular lenses among a plurality of automated stations for handling and processing the intraocular lenses to avoid the necessity for direct manual handling of the intraocular lenses. For example, the automated systems may include a conveyance or transport system for transporting and manipulating the intraocular lens including placing the intraocular lenses on multiple stations to process manufacturing orders (including identification of a lens model, cylinder power, etc.) In some embodiments, the conveyance system may include non-contact systems, such as a magnetic levitation transport systems, to reduce or eliminate debris due to friction or contact with other surfaces. As such, the automated systems may enable the intraocular lenses to be measured without physically damaging or contaminating the intraocular lenses, losing the intraocular lenses, or allowing the intraocular lenses to invert or roll over in a lens carrier.

Further, the automated systems may include embedded control loops enabling reactive corrections of environmental factors (e.g., temperature, water dosage, particle contamination etc.) and may enable product flows adjustment to account for out of calibration components and/or stations. The automated systems may also include control loops for product and data management (e.g., pass/fail analysis, handling re-measurements, handling measurements of multiple orders simultaneously, etc.). Additionally, the automated systems may quickly and efficiently identify the intraocular lenses that meet certain quality metrics and/or verification standards.

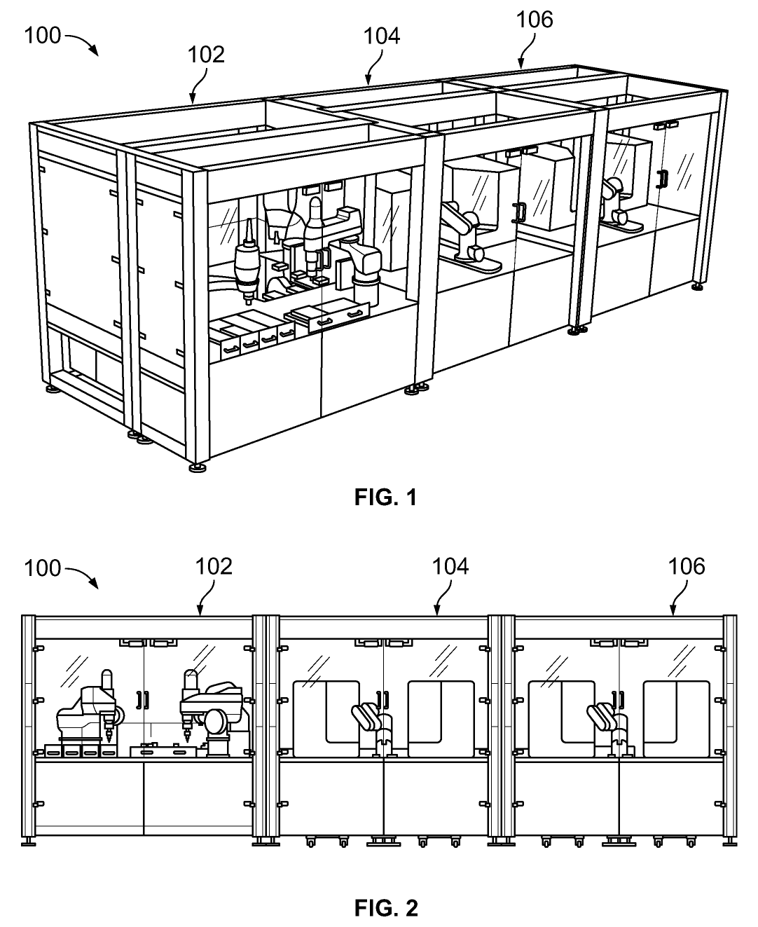

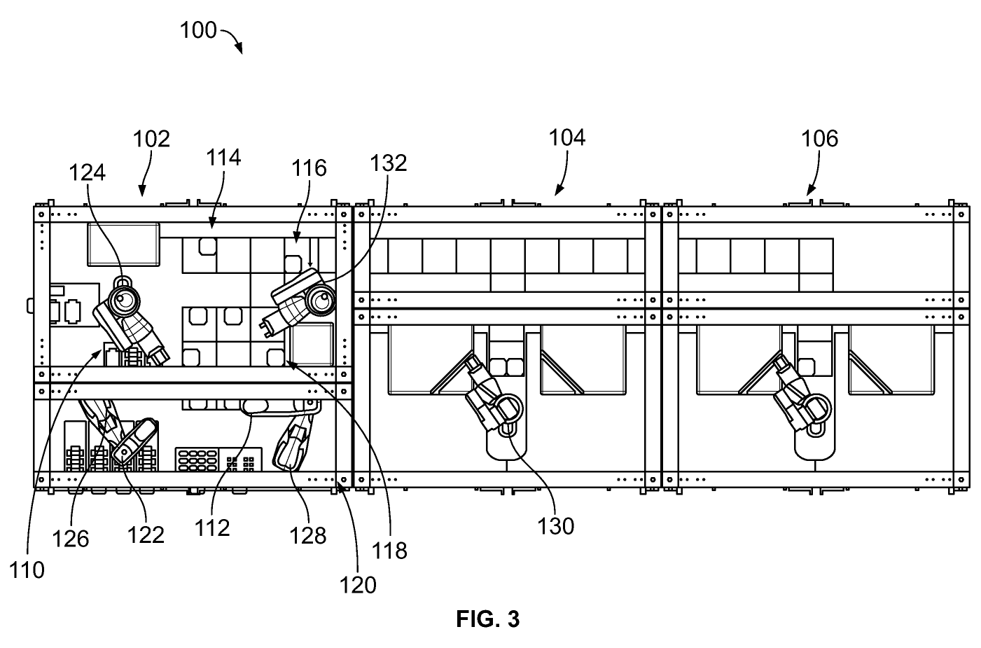

Referring now to the drawings, FIG. 1 illustrates a system 100 for performing metrology analysis on intraocular lenses (IOL), according to an exemplary embodiment. FIG. 2 shows a side elevational view of the system 100 and FIG. 3 shown a top view of the system 100. The system 100 may be capable of inspecting, measuring, and/or analyzing a wide variety of lens types, such as, for example, monofocal, spherical, toric, bifocal, extended depth of focus (EDOF),and multifocal lenses. For example, the system 100 may be configured to measure the optical characteristics and/or parameters of the intraocular lenses, such as, but not limited to, a modulation transfer function (MTF) and power measurements.

The system 100 includes a plurality of handling and/or processing stations designed to enable expedient and consolidated lens inspection and/or measurement processes. As shown in FIG. 3, the system 100 includes a handling module 102, and one or more metrology or measuring stations 104 and 106 (two being shown). The system 100 may include any suitable number of metrology stations. In some embodiments, the system 100 may include an automated inspection station for optical inspection, cosmetic inspection, or both. For example, the system 100 may include a cosmetic inspection station for inspecting the intraocular lenses (IOL) for cosmetic defects, such as flaws, tears, inclusions, and/or bubbles, as further described below. In some embodiments, one of the metrology stations 104 and 106 may comprise a cosmetic inspection station.

The handling module 102 of the system 100 includes a transfer station or area 110, a lens carrier assembly station or area 112, a dosing station or area 114, a purging station or area 116, a drying station or area 120, and an unloading station or area 122. The system 100 may also include a control system or unit (not shown) having one or more controllers or processors for controlling the operations of the handling module 102 and the metrology stations 104 and 106. The control system may be configured to track and/or trace an intraocular lens thru all the processing stations, collect data measurements of the intraocular lens at each of the processing station, and calibrate the processing stations.

Further, the control system of the system 100 may perform verification processes, based on verification standards, to ensure the system 100, including each of the processing and metrology stations, is suitable for use. For example, the control system may implement verification and/or diagnostic processes to test each of the stations, including the components and parts within the stations, to confirm that the stations of the system 100 are operating properly. The stations of the system 100 may also be individually tested to confirm that the stations are operating within predetermined tolerance levels. For example, the control system may verify that the measurements performed by the metrology stations are within acceptable tolerance ranges or levels. If tolerance levels of stations of the system 100 are satisfied, the system may be enabled for use. Without verifying that the system 100 is compliant with certain verification standards, there may be a risk for shifts or drifts in measurement results, decreasing throughput and increasing the failure rates of measured IOLs for modulation transfer functions (MTFs).

The handling module 102 of the system 100 may receive a tray or carrier container carrying one or more intraocular lenses. The tray may include holding or support elements or portions for holding the intraocular lenses and may have a single layer or multiple layers intraocular lenses. The tray may also include an identification element, such as a code or label (e.g., a bar code, a QR code, etc.) or an electronic device (e.g., an RFID tag). In some embodiments, the handling module 102 may be configured to identify each individual or separate lens. For example, each lens received by the handling module 102 may include an identification element.

The identification element may be used by the handling module 102 to identify and track the intraocular lenses at each of the processing stations of the system 100. The identification element may be associated with information or electronic data about the tray and/or the intraocular lenses carried by the tray. For example, the information associated with the identification element may include a unique identifier for the tray carrying the lenses, a quantity of intraocular lenses, a lens type of the intraocular lenses, a lot number and/or identifier (ID), a lot size, a product code, a lens carrier type, lens carrier components (e.g., a cup or container, a lid or top, and a lens holding assembly), lens parameters, and/or quality information. In some embodiments, the information about the tray and/or lens (e.g., quantity, tray number, etc.) may be manually input by an operator of the system 100 or retrieved from a database or memory of the system 100. The information may be communicated to each of the processing or handling stations of the system 100 as the intraocular lenses progress to each of the respective stations of the system 100.

The handling module 102 of the system 100 may include a reader or scanner (such as a bar code reader, a QR code reader, a radio frequency identification (RFID) reader, etc.) for detecting and scanning the identification element (such as, a bar code or a QR code) and/or for receiving a signal (e.g., a radio frequency identification signal) from the identification element of the tray. Based on the information obtained from the identification element, the handling module 102 may obtain or determine the type of the intraocular lenses carried by the tray. For example, the handling module 102 may compare the information obtained from the identification element to electronic data stored in the system 100 to determine the type of intraocular lenses. The handling module 102 may be configured to set or determine the measurements to be performed on the intraocular lenses in the metrology stations 104 and 106 according to the type of lens. The handling module 102 may also include a vision system or image capture system (e.g., a camera system) (not shown) that captures images of the intraocular lenses transported by the tray. In some embodiments, the automatic vision system may determine dimensions of the IOL, such as a diameter, haptic dimensions, etc. The vision system may determine the position of the intraocular lens in a coordinate system and may send the position coordinates and/or the dimensions to a robotic assembly 124 associated with the transfer station 110 for pick-up processing. For example, for toric IOLs, the vision system may detect alignment fiducials or alignment markings on the tray or on the lens itself.

As used herein, alignment fiducials (e.g. fiducial markers) refer to reference points or markers detectable and/or identifiable by the vision or camera system of the system 100 to determine position, location, and/or orientation information of the tray or other components or parts of the stations or system 100. In some embodiments, the alignment fiducials may include passive reflectors. The alignment fiducials may be placed at known locations on the tray or other components or parts of the stations or system 100. The alignment fiducials can be provided in any suitable manner, such as, but not limited to, a geometric shape of a portion of the tray. In some embodiments, two or more alignment fiducials may be configured in a known geometric relationship on the tray in any geometric shape. An arrangement of three alignment fiducials may be used to resolve all six degrees of freedom (6-DOF) of the tray or other components or parts of the stations or system. The alignment fiducials may be captured by the vision system of the system 100 within an image and the location of the alignment fiducials may then be identified and used to determine where the tray or other parts of the stations or system are within an environment relative to the vision system. For instance, one or more alignment fiducials on the tray may be used to determine the (X, Y, Z) position of the tray is relative to the vision system.

Based on the alignment fiducials detected by the vision system, the vision system may calculate the position of the intraocular lenses on the tray for enabling the intraocular lenses to be picked up by the robotic assemblies of the system 100 for subsequent optical measurements (i.e. diopter power, cylinder, image quality, alignment of fiducial markings with the low power meridian etc.). For example, once the robotic assembly 124 receives the position information from the vision system, the robotic assembly 124 may select or pick-up one of the intraocular lenses from the tray and place the lens in a lens holding assembly as further described below.

A robotic assembly 126 of the system 100 may be configured to select components for a lens carrier or a water cell from an input tray. The lens carrier components may be used to construct lens carriers for holding the intraocular lenses while the lens carrier is moved between the various processing or handling stations of the system 100. The lens carrier components may include alignment markings or alignment fiducials to enable the robotic assembly 126 to determine the position of the lens carrier components and pick up the lens carrier components. The robotic assembly 126 of may place the selected components for the lens carrier on an intermediate assembly station.

Once the robotic assembly 126 selects the lens carrier components, a robotic assembly 128 may be configured to construct the lens carrier. The alignment fiducials of the lens carrier components may enable the system 100 to determine the location of the components for the lens carrier in order to facilitate the assembly of the lens carrier. The lens carrier may be configured to hold the lens holding assembly that carries an intraocular lens. For example, the lens holding assembly, along with the intraocular lens, may be positioned within the lens carrier. Once placed within the lens carrier, the intraocular lens may be immersed in a solution or fluid to enable the intraocular lens to be analyzed and/or measured using metrology equipment, such as an interferometer. In some embodiments, the lens carrier may include a cup or container and a lid or cover. The lid may have an opening to enable measurement equipment to measure the intraocular lens without removing the lid from the container. As such, the lens holding assembly may be configured to be positioned within a cavity of the container of the lens carrier. It is contemplated that the lens carrier may take any suitable form. For example, the lens carrier may be designed to accommodate multiple intraocular lenses.

When assembling the lens carrier, the robotic assembly 128 may be configured to select the lens holding assembly that holds the intraocular lens. The lens holding assembly may be placed by the robotic assembly 128 in the container of the lens carrier. In some embodiments, the robotic assembly 128 may flip or invert the lens holding assembly (and the intraocular lens held by the assembly) and then place the flipped lens holding assembly in the container of the lens carrier. Subsequently, the robotic assembly 128 may place the lid on the lens carrier to complete the assembly of the lens carrier. The lens carrier may include an identification element (e.g., a bar code, a QR code, a RFID tag, etc.) that may be read or scanned by an identification (ID) reader (e.g., a bar code reader, a RFID reader, etc.). The data collected by the ID reader may be used to track the lens carrier and the associated intralocular lens as the lens progresses to various processing or handling stations of the system 100.

Once the assembly of the lens carrier is completed, the robotic assembly 128 may be configured to move the completed lens carrier to a conveyance or transport system that automatically moves the lens carrier to the dosing station 114 of the handling module 102, where a solution or fluid is dispensed into the lens carrier. The conveyance system may comprise a non-contact system, such as a magnetic levitation transport system, to reduce or eliminate debris due to friction or contact with other surfaces.

Once the lens carrier is received at the dosing station 114 of the handling module 102, the dosing station 114 may be configured to fill the lens carrier with a solution or fluid, such as deionized (DI) water. While the lens carrier is filled with the solution, the dosing station 114 may hold the lens carrier at a particular inclination angle to facilitate the displacement of air within the lens carrier.

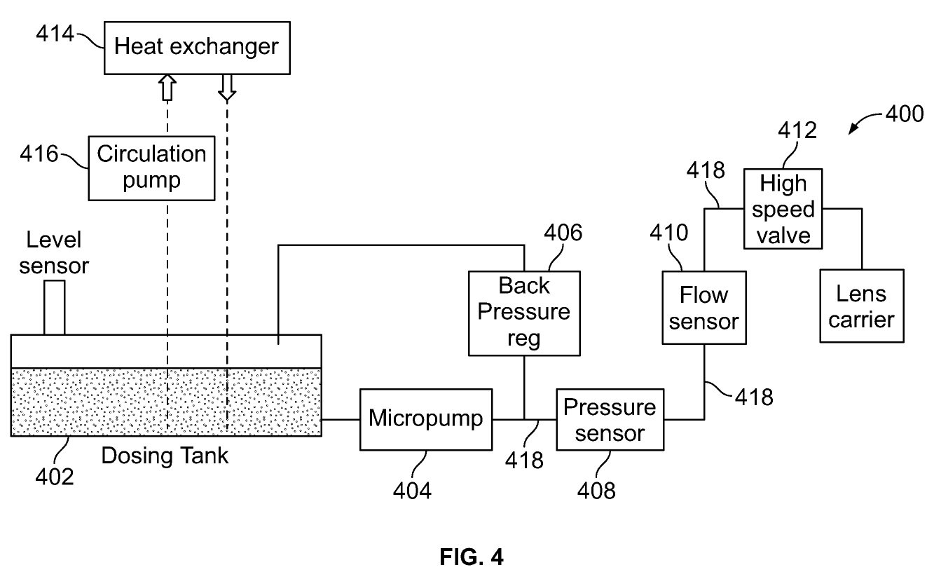

In some embodiments, the dosing station 114 may be configured to cool the solution used to fill the lens carrier to a predetermined temperature. FIG. 4 shows a block diagram of an exemplary dosing station 400. As shown in FIG. 4, the exemplary dosing station 400 includes a dosing tank or fluid reservoir 402, a micropump 404, a back pressure regulator 406, a pressure sensor 408, a flow sensor 410, and a high speed valve or solenoid valve 412. The exemplary dosing station 400 may also include a heat exchanger 414 and/or a circulation pump 416.

In some embodiments, the dosing station 114 may be configured to perform a sanitation function by heating the solution used to fill the lens carrier to a predetermined temperature. By way of example this heated temperature may be up to 50° F, up to 60° F, up to 70° F, up to 80° F, or over 80° F. After such heating, the dosing station may then be configured as described to adjust the temperature for optimal use in the system.

The dosing tank 402 of the exemplary dosing station 400 may hold a solution or fluid for filling the lens carrier. The circulation pump 412 may be configured to pull the solution from the dosing tank 402 and circulate the fluid through the heat exchanger 414 to remove heat from the fluid. In some embodiments, a chiller (not shown) may be configured to circulate a liquid through the heat exchanger 414 to facilitate the cooling of the temperate of the solution. After the solution is circulated through the heat exchanger 414, the solution may be returned to the dosing tank 402.

The temperature of the solution in the dosing tank 402 may be maintained at a predetermined temperature. For example, the temperature of the solution in the dosing tank 402 may be controlled by automatically adjusting the temperature of a chiller and/or controlling the flow rate of the solution through the heat exchanger 414 by adjusting the speed of the circulation pump 414. Sensors, such as temperature and flow sensors, may be used to measure the temperature of the solution in the dosing tank 402.

To dispense the solution held by the dosing tank 402 into the lens carrier, the micropump 404 of the exemplary dosing station 400 may pull the solution from the dosing tank 402 and may circulate the solution through a line or conduit 418 and into the lens carrier. In some embodiments, the micropump 404 may be configured to be constantly running. As shown in FIG. 4, the pressure sensor 408, the flow sensor 410, and the high-speed valve 412 may be coupled to the line 418. The back-pressure regulator 406 may maintain constant pressure between the micropump 404 and the high-speed valve 412.

Once the lens carrier is filled with the solution or fluid (e.g., DI water), the dosing station 400 may perform a temperature check of the solution in the lens carrier to ensure the temperature is within an acceptable temperature range. The temperature check may be configured as a close loop temperature control to ensure the solution in the lens carrier is at an optical temperature right before measurements are performed on the lens. In addition, the dosing station 400 may measure or determine the temperature of the intraocular lens to ensure the temperature of the intraocular lens is at or near a predetermined temperature.

After performing the temperature check, the control system of the system 100 may determine the availability of the metrology stations 104 and 106 and select one of the metrology stations 104 and 106 to receive the lens carrier. After a metrology station in selected, the conveyance system may be configured to move the lens carrier to the selected metrology station 104 or 106. For example, when the metrology station 104 is selected to receive the lens carrier, the lens carrier may be transferred to the metrology station 104. A robotic assembly 130 associated with the metrology station 104 may be configured to transfer the lens carrier to the measuring equipment of the metrology station 104.

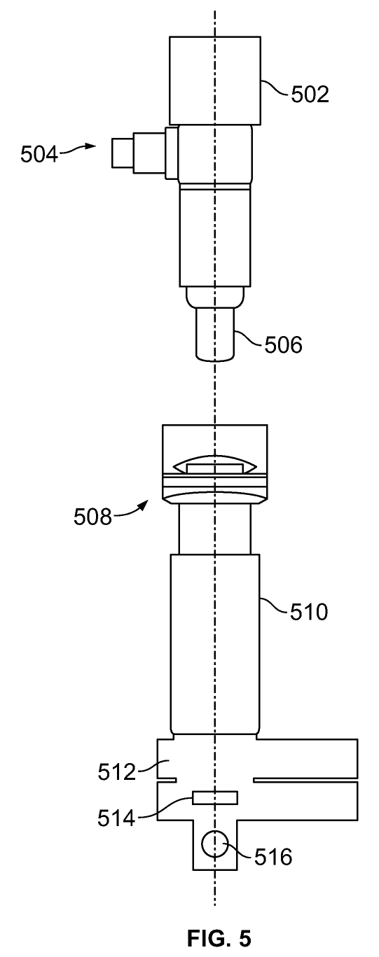

FIG. 5 shows a schematic diagram of exemplary embodiment of the measuring equipment of the metrology station 104. As shown in FIG. 5, the metrology station 104 includes a vision or camera system 502, a light source 504, a microscope 506, an aperture wheel 508, a collimator 510, a reticle changer 512, a filter 514, and a light source 516. It will be recognized that the metrology station 104 may have any suitable configuration to measure the characteristics of a lens.

The vision system 502 of the metrology station 104 may capture images of an intraocular lens held by the lens holding assembly. The vision system 502 may detect alignment markings or alignment fiducials of the lens carrier to determine the location of the lens carrier for positioning the lens carrier at a desired position within the metrology station 104. The vision system may also detect alignment markings or alignment fiducials of the lens holding assembly held by the lens carrier for determining the position of the lens holding assembly. Based on the alignment fiducials of the lens holding assembly detected by the vision system 502, the vision system 502 may calculate the position of the intraocular lens.

After the position of the intraocular lens is determined, the measuring equipment of the metrology station 104 may be configured to perform measurements for determining a modulation transfer function (MTF) of the intraocular lens. In some embodiments, the measuring equipment may perform other lens measurements to determine a variety of parameters, such as, for example, power measurements (sphere, cylinder, etc.), an effective focal length (EFL), a point spread function (PSF and LSF), a MTF focus scan (“through focus scan“), an energy distribution (MTF area), imaging quality, Strehl ratio, transmitted wavefront, etc.

When the metrology station 106 of the system 100 is selected to receive the lens assembly, the lens assembly may be transferred to the metrology station 106. In some embodiments, the metrology station 106 of the system 100 may be configured to perform measurements and functions similar to the measurements and functions performed by the metrology station 104. For example, a robotic assembly associated with the metrology station 106 may be configured to transfer the lens carrier to the measuring equipment of the metrology station 106. The measuring equipment may be configured to perform measurements to determine a modulation transfer function (MTF) of the intraocular lens and/or any other optical or dimensional characteristic of the intraocular lens. The measuring equipment of the metrology station 106 may be constructed and function in a similar manner as the measuring equipment of the previously described metrology station 104 of FIG. 5.

In some embodiments, the metrology stations 104 and 106 may perform the same or similar measurement on the intraocular lens for comparison purposes. In other embodiments, the metrology stations 104 and 106 may perform different measurements or processes on the intraocular lens. For example, one of the metrology stations 104 or 106 may comprises an automated inspection station for optical inspection, cosmetic inspection, or both, as further described below. The lens carrier may be received and processed by one or both of the metrology stations 104 and 106 depending on the configuration and the system requirements.

After the measurements are completed by the metrology station 104 and/or the metrology station106, a robotic assembly of the metrology stations may transfer the lens carrier to the conveyance system. The conveyance system may then automatically deliver the lens carrier to the purging station 116 of the handling module 102. The purging station 116 of the handling module 102 may be configured to remove the solution (e.g., DI water) from the lens carrier. In some embodiments, the purging station 116 may tilt and/or hold the lens carrier at a predetermined angle to facilitate the removal of the solution from the lens carrier. The purging station 116 may discharge the solution into to a waste reservoir.

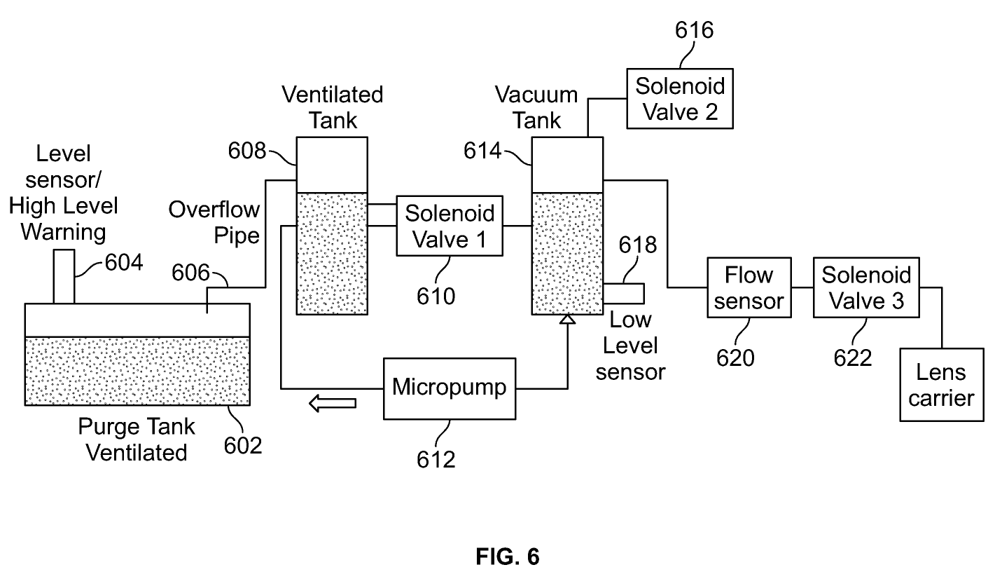

FIG. 6 shows a block diagram of an exemplary purging station or system 600. As shown in FIG. 6, the purging station or system 600 includes a purging tank 602, a level sensor 604, an overflow pipe 606, a ventilated tank 608, a solenoid valve 610, a micropump 612, a vacuum tank 614, a solenoid valve 616, a low-level sensor 618, a flow sensor 620, and a solenoid valve 622. The purging station 600 have an idle mode and a purging mode. In the idle mode, the solenoid valve 610 and solenoid valve 616 are opened and the solution re-circulates between the vacuum tank 614 and the ventilated tank 608. The solenoid valve 616 allows the ventilated tank 608 to vent to the atmosphere. In the purge mode, the solenoid valve 610 and solenoid valve 616 are closed and a nozzle is placed in the lens carrier. The micropump 612 may be active to create a slight vacuum in the vacuum tank 614. The solenoid valve 622 may be opened and the solution may be removed from the lens carrier. Once the solution is removed, the solenoid valve 622 is closed and solenoid valves 610 and 616 are opened to return the system to the idle mode. When the level of the solution in the purging tank 602 exceeds a high level or threshold, the level sensor 604 may emit an alarm.

After removing the solution from the lens carrier at the purging station 600, the lens carrier may be moved by the conveyance system to the drying station 120 of the handling module 102. The drying station 120 of the handling module 102 may be configured to dry the lens holding assembly of the lens carrier. At the purging station 600, a robotic assembly 132 may be configured to disassemble the lens carrier. For example, the robotic assembly 132 may remove the lid from the container of the lens carrier. The robotic assembly 132 may then be configured to remove the lens holding assembly that holds the intraocular lens from the lens carrier and place the lens holding assembly at the drying station 120. The drying station 120 may consist of a rotary base that receives the lens holding assembly in a vertical orientation. The drying station 120 may be capable of rotating the lens holding assembly while one or more air nozzles inject air to the lens holding assembly. The air may force the remaining water out of the surfaces of the lens holding assembly.

Once the lens holding assembly is dried and oriented, the lens holding assembly may be passed to an automated inspection station for optical inspection, cosmetic inspection, or both. For example, the automatic inspection station may include a cosmetic inspection station for inspecting the intraocular lenses (IOL) for cosmetic defects, such as flaws, tears, inclusions and/or bubbles. The cosmetic inspection station may also include an automatic vision system or image capture system (e.g., a camera system) that captures images of the lens holding assembly. The vision system may detect alignment fiducials of the lens holding assembly to determine the position of the lens holding assembly and to place the lens holding assembly at a desired position for inspection. Based on the position of the detected alignment fiducials of the lens holding assembly, the position of the intraocular lens may be calculated for performing an inspection of the lens. For example, an image of the alignment fiducials of the lens holding assembly may be captured by the vision system to determine the position of the intraocular lens.

After the intraocular lens is inspected, the lens carrier may be passed to the unloading station 122, The unloading station 122 may be configured to remove the intraocular lenses that failed the inspection process or a verification process based on the measurements performed at the metrology stations 104 and 106. For example, the unloading station 122 may be configured to determine whether or not the measured characteristics of the intraocular lens falls within a predetermined allowable range and/or meets a particular verification standard. In some embodiments, the unloading station 122 may determine whether the measured MTF of the intraocular lens is within an allowable range. When the MTF of the intraocular lens is not within the allowable range, the intraocular lens may be rejected and the robotic assembly 124 may place the intraocular lens in a rejected container or tray. When the measurements of the intraocular lens fall within the allowable range, the robotic assembly 124 may place the intraocular lens in a “passed” container or tray. The process described above may be repeated for each intraocular lens that is in the tray or carrier container received by the handling module 102 of the system.

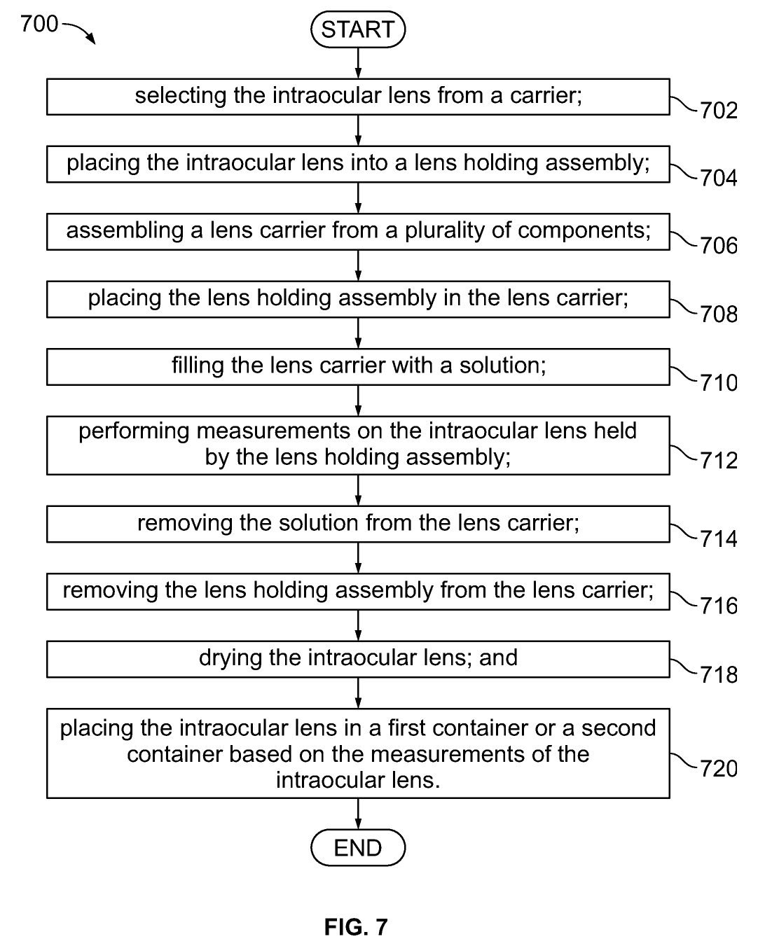

FIG. 7 is a block diagram of an exemplary process 700 for performing measurement on an intraocular lens. At block 702, the process involves picking up the intraocular lens from a carrier. For example, a robotic assembly (e.g., robotic assembly 124) may be configured to pick up the intraocular lens from a tray. At block 704, the process involves placing the intraocular lens into a lens holding assembly. For example, a robotic assembly (e.g., robotic assembly 124) and place the intraocular lens into a lens holding assembly. At block 706, the process involves assembling a lens carrier from a plurality of components. In some examples, the lens carrier may be assembled from a first part (e.g., holder or container), a second part (e.g., a top or lid), and the IOL. For example, the lens carrier may include a cup or container and a lid or cover. It is contemplated that the lens carrier may take any suitable form.

The lens holding assembly may be picked up by a robotic assembly and placed in the lens carrier at block 708. In some embodiments, the robotic assembly may flip or invert the lens holding assembly (and the intraocular lens held by the assembly) and then place the flipped lens holding assembly in the lens carrier. At block 710, the process involves filling the lens carrier with a solution.

At block 712, the process involves performing measurements on the intraocular lens. For example, a metrology station (e.g., metrology station 106) may perform measurements on the intraocular lens. In some embodiments, the metrology station may be configured to performed measurements to determine a modulation transfer function (MTF) of the intraocular lens or any other suitable measurement.

At block 714, the process involves removing the solution from the lens carrier. At block 716, the process involves removing the lens holding assembly from the lens carrier, and at block 720, the process involves drying the intraocular lens. In some embodiments, the process may include an optical inspection, cosmetic inspection, or both. For example, the process may include an inspection for inspecting the intraocular lens for cosmetic defects, such as flaws, tears, inclusions, and/or bubbles. At block 720, the process involves placing the intraocular lens in a first or second container based on the measurements and/or inspection of the intraocular lens.

The above description is given for purposes of illustration and explanation. It will be apparent to those skilled in the relevant art that changes and modifications may be made to the embodiments described above without departing from its scope or spirit.

What is claimed is:

1. An automated apparatus for measuring characteristics of an intraocular lens comprising:
a first robotic assembly configured to select the intraocular lens from a tray and to place the intraocular lens into a lens holding assembly;
a second robotic assembly configured to assemble a lens carrier from a plurality of components, wherein the second robotic assembly places the lens holding assembly in the lens carrier;
a transport system for transporting the lens carrier to and from a plurality of stations, wherein the lens carrier is placed on the transport system by the second robotic assembly;
a first automated station for receiving the lens carrier and filling the lens carrier with a solution;
a second automated station for receiving the lens carrier and performing measurements on the intraocular lens carried by the lens carrier;
a third automated station for removing the solution from the lens carrier;
a third robotic assembly configured to remove the lens holding assembly from the lens carrier; and
a fourth automated station configured to receive the lens holding assembly from the third robotic assembly and to dry the intraocular lens.

2. The automated apparatus of claim 1, wherein the first automated station comprises a dosing station, wherein the second automated station comprises metrology station, wherein the third automated station comprising a purging station, and wherein the fourth automated station comprises a drying station.

3. The automated apparatus of claim 1, further comprising at least one controller for controlling the operations of the automated stations and the robotic assemblies.

4. The automated apparatus of claim 1, wherein the second robotic assembly is configured to flip or invert the lens holding assembly prior to placing the lens holding assembly in the lens carrier.

5. The automated apparatus of claim 1, wherein the lens carrier maintains an orientation of the intraocular lens without permitting the intraocular lens to invert or be displaced in the measurement axis or roll over.

6. The automated apparatus of claim 1, further comprising a fourth robotic assembly, wherein the fourth robotic assembly is configured to select components for the lens carrier and to place the components on an intermediate assembly station.

7. The automated apparatus of claim 1, wherein the intraocular lens is placed on a top portion of the lens holding assembly by the first robotic assembly.

8. The automated apparatus of claim 7, wherein the second robotic assembly is configured to place a bottom portion of the lens holding assembly on the top portion of the lens holding assembly, and wherein the second robotic assembly is further configured to place a lid or top on the lens carrier.

9. The automated apparatus of claim 1, wherein the first automated station is configured to hold the lens carrier at a predetermined angle to facilitate displacement of air within the lens carrier while the solution is dosed.

10. The automated apparatus of claim 1, wherein the second automated station is configured to determine a modulation transfer function (MFT) of the intraocular lens, a power measurement, an effective focal length, a point spread function, a MTF focus scan, an energy distribution, imaging quality, a Strehl ratio, a transmitted wavefront, or a combination thereof.

11. The automated apparatus of claim 1, further comprising a fifth robotic assembly, wherein the fifth robotic assembly is configured to transfer the lens carrier from the transport system and to place the lens carrier at metrology equipment of the second automated station.

12. The automated apparatus of claim 1, wherein the third automated station is configured to hold the lens carrier at a predetermined angle to facilitate removal of the solution.

13. The automated apparatus of claim 2, wherein the dosing station is configured to adjust a temperature of the solution to a predetermined temperature.

14. The automated apparatus of claim 1, wherein the first automated station is configured to measure a temperature of the solution or the intraocular lens and compare the temperature to a temperature threshold.

15. The automated apparatus of claim 1, wherein the second robotic assembly is configured to remove the lens holding assembly from the lens carrier and to place the lens holding assembly at the fourth automated station.

16. The automated apparatus of claim 15, wherein the fourth automated station is configured to rotate the lens holding assembly to facilitate the drying of the intraocular lens.

17. The automated apparatus of claim 16, wherein the first robotic assembly is configured to remove the intraocular lens from the lens holding assembly and to place the intraocular lens in a pass container or a reject container based on measurements of the intraocular lens or verification standards.

18. The automated apparatus of claim 1, further comprising a fifth automated station, wherein the fifth automated station is configured to inspect the intraocular lens for cosmetic defects.

19. The automated apparatus of claim 1, further comprising a vision system, wherein the vision system is configured to detect one or more alignment fiducials to determine a position of the lens carrier, the lens holding assembly, or the intraocular lens.

20. The automated apparatus of claim 1, wherein the transport system comprises a magnetic levitation transport system.

21. A method for performing measurements on an intraocular lens comprising:
selecting the intraocular lens from a carrier;
placing the intraocular lens into a lens holding assembly;
assembling a lens carrier from a plurality of components;
placing the lens holding assembly in the lens carrier;
filling the lens carrier with a solution;
performing measurements on the intraocular lens carried by the lens carrier;
removing the solution from the lens carrier;
removing the lens holding assembly from the lens carrier;
drying the intraocular lens; and
placing the intraocular lens in a first container or a second container based on the measurements of the intraocular lens.

22. The method of claim 21, wherein the lens holding assembly is flipped or inverted prior to placing the lens holding assembly in the lens carrier.

23. The method of claim 21, further comprising selecting components for the lens carrier and placing the components on an intermediate assembly station.

24. The method of claim 21, further comprising measuring characteristics of the intraocular lens to determine a modulation transfer function, a power measurement, an effective focal length, a point spread function, a MTF focus scan, an energy distribution, imaging quality, a Strehl ratio, a transmitted wavefront, or a combination thereof.

25. The method of claim 21, further comprising identifying a type or model of the intraocular lens using a vision system.

26. The method of claim 21, further comprising adjusting a temperature of the solution to a predetermined temperature.

27. The method of claim 21, further comprising measuring a temperature of the solution or the intraocular lens and comparing the temperature to a threshold.

28. The method of claim 21, further comprising tracking the intraocular lens during the measurement process.

29. The method of claim 21, performing an inspection of the intraocular lens to detect cosmetic defects or performing a verification process.

30. The method of claim 21, further comprising detecting one or more alignment fiducials to determine a position of the lens carrier, the lens holding assembly, or the intraocular lens.

31. The method of claim 21, further comprising transporting the lens carrier to one or more processing station using a magnetic levitation transport system.
